(19) United States
(12) Patent Application Publication
(10) Pub. No.: US 2026/0283810 A1
(43) Pub. Date: Sep. 24, 2026

(54) ORTHOPAEDIC IMPLANT SYSTEMS INCLUDING RETENTION FEATURES AND METHODS OF REPAIR

(71) Applicant: ARTHREX, INC., Naples, FL (US)

(72) Inventors: Rudraksh Khosla, Naples, FL (US); Gregory Guederian, Naples, FL (US); Jason Schultz, Naples, FL (US); Jerry Sterrett, Bonita Springs, FL (US); John David Paterson, Naples, FL (US)

(21) Appl. No.: 19/569,128

(22) Filed: Mar. 17, 2026

Related U.S. Application Data
(60) Provisional application No. 63/773,675, filed on Mar. 18, 2025.

(51) Int. Cl. A61F 2/40; A61F 2/30; A61F 2/46

(52) U.S. Cl. CPC A61F 2/4014; A61F 2/4059; A61F 2/4684; A61F 2002/30327; A61F 2002/30331; A61F 2002/30433; A61F 2002/4022; A61F 2002/4033; A61F 2002/4037; A61F 2002/4044

(57) ABSTRACT

This disclosure relates to orthopaedic implant systems and methods for restoring functionality to a bone and/or joint. The implant systems disclosed herein may include one or more retention features adapted to contact a bone, such as one or flanges. The flange may be deflectable and may be compressed against the bone. A method of installing an orthopaedic implant system is also disclosed.

### CROSS-REFERENCE TO RELATED APPLICATIONS

This application claims the benefit of U.S. Provisional Application No. 63/773,675, filed Mar. 18, 2025, which is incorporated herein by reference in its entirety.

### BACKGROUND

This disclosure relates to orthopaedic procedures and, more particularly, to orthopaedic implant systems and methods for repairing bone defects and restoring functionality to a joint.

Joints may be established by at least one long bone. The patient may experience loss caused by loosening of a previously implanted prosthesis or fracturing or shattering of the bone due to trauma. A prosthesis may be utilized to restore functionality to the bone. The prosthesis may include a stem insertable in the bone and may include a head for mounting an articulation member to restore functionality to a joint.

### SUMMARY

This disclosure relates to orthopaedic implant systems and methods for restoring functionality to a joint. The implant systems may include one or more retention features such as one or more flanges adapted to contact a bone, which may be associated with a joint. The flange may be deflectable and may be compressed against the bone.

A system for an orthopaedic procedure according to an implementation may include a head portion adapted to mount an articulation member. The articulation member may be adapted to mate with an opposed articular surface. A stem portion may be insertable into bone. The stem portion may include a stem body extending along an axis. A collar portion may include a collar body and at least one deflectable flange that may extend from the collar body to a free end. The collar body may be adapted to interconnect the head portion and the stem portion in an assembled configuration. The at least one flange may be dimensioned to abut bone.

A system for an orthopaedic procedure according to an implementation may include a stem portion insertable into bone. The stem portion may include a stem body extending along an axis. A collar portion may include a collar body and at least one flange dimensioned to abut bone. The collar body may extend between a first end and a second end. The collar body may be securable to the stem portion at a second interface adjacent to the second end in an assembled configuration. The at least one flange may include a ramp adapted to cause the at least one flange deflect outwardly in a first direction in response to engagement with the bone. The at least one flange may be compressible in a second direction against a periphery of the bone subsequent to the deflection.

A system for an orthopaedic procedure according to an implementation may include a head portion adapted to mount an articulation member. The articulation member may be adapted to mate with an opposed articular surface. A stem portion may be insertable into bone. The stem portion may include a stem body extending along an axis. A collar portion may include a collar body adapted to interconnect the head portion and the stem portion in an assembled configuration. The collar portion may include a plurality of protrusions extending from a distal end of the collar body. The protrusions may be engageable with bone to limit relative rotation between the collar portion and the bone.

A method of installing an orthopaedic implant system according to an implementation may include coupling a stem portion and a collar portion. The collar portion may include at least one flange. The method may include inserting the stem portion into an intramedullary canal of a bone. The method may include trapping a cortical wall of the bone between the at least one flange and the stem portion in response to compressing the at least one flange against the bone. The method may include coupling a head portion and the collar portion such that the collar portion may interconnect the stem portion and the head portion. The method may include mounting an articulation member to the head portion. The articulation member may mate with an opposed articular surface.

The present disclosure may include any one or more of the individual features disclosed above and/or below alone or in any combination thereof.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 discloses a surgical suite according to an implementation.

FIG. 2 discloses a perspective view of an implant system including a collar portion having a flange.

FIG. 3 discloses a sectional view of the implant system of FIG. 2.

FIG. 4 discloses an exploded view of the implant system of FIG. 3.

FIG. 5 discloses another sectional view of the implant system of FIG. 2.

FIG. 6 discloses a sectional view of a portion of the implant system of FIG. 2 positioned relative to a bone.

FIG. 7 discloses a perspective view of the implant system including an articulation member.

FIG. 8 discloses a side view of the articulation member of FIG. 7.

FIG. 9 discloses a side view of an implant system including a set of flanges according to another implementation.

FIG. 10 discloses the implant system of FIG. 9 positioned relative to a bone.

FIG. 11 discloses a perspective view of the implant system of FIG. 10 and a surgical instrument.

FIG. 12 discloses an implant system associated with a flexible construct according to another implementation.

FIG. 13 discloses a sectional view of an implant system including a collar portion having a set of flanges according to another implementation.

FIG. 14 discloses an isolated view of a collar portion of the implant system of FIG. 13.

FIG. 15 discloses a perspective view of an implant system including a collar portion having a set of flanges according to another implementation.

FIG. 16 discloses a perspective view of an implant system including engagement features according to an implementation.

FIG. 17 discloses the implant system of FIG. 16 positioned relative to a bone.

FIG. 18 discloses a method of installing an orthopaedic implant system.

FIG. 19 discloses a surgical instrument and removal of bone according to an implementation.

FIG. 20 discloses a surgical instrument and removal of bone according to another implementation.

FIG. 21 discloses placement of a trial according to an implementation.

Like reference numbers and designations in the various drawings indicate like elements.

### DETAILED DESCRIPTION

This disclosure relates to orthopaedic implant systems (e.g., assemblies) and methods for repairing bone defects and restoring functionality to various joints. The disclosed implant systems described herein may include a modular prosthesis that may be adapted to facilitate reconstruction or repair of a long bone that may be caused by trauma to the patient. The long bone may have relatively severe proximal bone loss due to fracturing (e.g., 3 part or 4 part fractures) or shattering of the bone due to trauma, or a tumor in the bone. The disclosed implant systems and methods may be utilized to restore functionality to a shoulder joint, including anatomical and reverse shoulder procedures.

Arthroplasty systems may rely on proximal humeral fixation. Over time, the humeral implant may fail due to severe proximal humeral bone loss. Other arthroplasty systems may utilize distal humeral fixation. Torsional loosening of a stem of the implant may occur due to relatively high torsional forces, which may cause a twisting action between an articulation member (e.g., proximal body or head) and the stem. The surgeon may resect and remove the diseased proximal bone and replace it with an implant secured to the bone, such as the distal humerus. The disclosed techniques may be utilized to resist torsional forces on the stem that may otherwise cause loosening in a press fit application.

The disclosed implant systems may include a collar having one or more retention features. The retention features may include one or more flanges. The collar may include various numbers of flanges, such as a single flange, a dual flange arrangement, or a triple flange arrangement. Incorporating a set of flanges may improve stability. The flanges may be adapted to engage an adjacent bone, such as the distal humerus. The flange may be adapted to deflect outwardly in response to contact with the bone, which may be suitable for accommodating various anatomies. The anatomical profile of the bone may be generally circular (e.g., proximal humeral shaft) or may be generally triangular (e.g., distal humeral shaft). The flanges may be compressed against the bone utilizing various techniques, including one or more fasteners (e.g., screws), flexible constructs (e.g., suture, wire or cable), or a cam (e.g., clamp). The flexible construct may be wrapped around the flange and bone and may be tensioned. The cam may be adapted to clamp onto a periphery of the bone.

A fastener may be passed through the flange and stem, which may cause a head of the fastener to bend the flange allowing it compress against an external surface of the bone, such as a cortical wall. The flanges may be compressed to provide tension against the external surface of the bone. In implementations, the flanges may be omitted.

The collar may include one or more engagement features adapted to engage the bone. The engagement features may be limit relative rotation between the collar and the stem. The engagement features may be distributed along the flange and/or a bottom (e.g., distal end) of the collar. The engagement features may include protrusions (e.g., spikes) and/or plasma spray to limit relative rotation between the collar and the stem. The engagement features may be embedded into the adjacent bone. The engagement features may increase friction with the bone and may promote osseointegration of bone between the engagement features. Omitting the flanges and incorporating engagement features (e.g., protrusions or spikes) along the bottom of the collar may provide a low profile solution. The engagement features may improve torsional stability of the implant system.

The implant system may include a press-fit distal stem. The stem may include one or more holes to allow passage of fasteners (e.g., screws) through the stem. A collar may be coupled to the stem utilizing various techniques, such as a taper connection. A bottom (e.g., distal end) of the collar may be positioned to rest on a resected bone. The stem and/or bottom of the collar may be treated with plasma spray for osseointegration. One or more segments (e.g., spacers or extensions) may be assembled onto the collar utilizing various techniques, including a taper connection. The segments may be assembled to bridge the space between the articulation member and the collar. The segments may incorporate suture holes and/or may be treated with plasma spray to facilate soft tissue attachment.

A proximal component (e.g., head) may be adapted to mount the articulation member, which may include a reverse liner. The surgeon may be provided proximal components having different lateral widths based on anatomy to optimize soft tissue tensioning for reducing instability. The proximal component may incorporate suture holes and/or may be treated with plasma spray to facilate soft tissue attachment.

A system for an orthopaedic procedure according to an implementation may include a head portion adapted to mount an articulation member. The articulation member may be adapted to mate with an opposed articular surface. A stem portion may be insertable into bone. The stem portion may include a stem body extending along an axis. A collar portion may include a collar body and at least one deflectable flange that may extend from the collar body to a free end. The collar body may be adapted to interconnect the head portion and the stem portion in an assembled configuration. The at least one flange may be dimensioned to abut bone.

In any implementations, the free end may be aligned with the stem body relative to the axis in the assembled configuration.

In any implementations, the at least one flange may include a plurality of flanges distributed about a periphery of the collar body.

In any implementations, the plurality of flanges may include first and second flanges on opposite sides of the collar body.

In any implementations, the collar body may extend between a proximal end and a distal end. The collar body may establish an interface with the stem portion adjacent to the distal end in the assembled configuration.

In any implementations, the free end of the at least one flange may be distal to the distal end of the collar body in the assembled configuration.

In any implementations, a perimeter of the collar body at the distal end may be greater than a perimeter of the stem body aligned with the distal end such that a portion of the distal end may be adapted to sit on a resected bone surface in the assembled configuration.

In any implementations, the collar body may include one or more protrusions along the distal end. The one or more protrusions may be engageable with bone to limit relative rotation between the collar portion and the bone.

In any implementations, the at least one flange may include a first flange segment and a second flange segment. The first flange segment may extend radially outwardly in a first direction from a first position along a periphery of the collar body to establish a separation gap between the first flange segment and the periphery of the collar body. The second flange segment may extend radially inwardly in a second direction from the first flange segment towards the free end to establish an engagement surface that may be dimensioned to face towards the stem portion in the assembled configuration. The first and second directions may be relative to the axis.

In any implementations, the at least one flange may include a third flange segment that establishes the free end. The second flange segment may interconnect the first and third flange segments. The third flange segment may extend radially outwardly in a third direction relative to the axis. The third flange segment may include a ramped engagement surface that may slope outwardly from the engagement surface of the second flange segment. The ramped engagement surface may be dimensioned to cause the at least one flange to deflect outwardly in response to engagement with bone.

In any implementations, the collar body may extend between a proximal end and a distal end. The first position of the first flange segment may be spaced apart from the proximal and distal ends of the collar body.

In any implementations, the collar portion may include a recess in the periphery of the collar body that may extend distally from the first position along the separation gap.

In any implementations, the engagement surface may include one or more protrusions engageable with bone to limit relative rotation between the collar portion and the bone.

In any implementations, the at least one flange may be adapted to deflect inwardly toward the collar body in response to a compressive force.

In any implementations, the at least one flange may include an aperture dimensioned to receive a fastener that establishes the compressive force.

In any implementations, the stem portion may include a stem passage spaced apart from the collar portion in the assembled configuration. The stem passage may be dimensioned to receive a portion of the fastener in the assembled configuration.

In any implementations, the at least one flange may include one or more recesses dimensioned to receive a flexible construct. The flexible construct may be dimensioned to loop about the axis to establish the compressive force.

In any implementations, the flexible construct may include a length of suture or a length of wire.

In any implementations, the collar body and the stem body may establish a taper connection in the assembled configuration.

A system for an orthopaedic procedure according to an implementation may include a stem portion insertable into bone. The stem portion may include a stem body extending along an axis. A collar portion may include a collar body and at least one flange dimensioned to abut bone. The collar body may extend between a first end and a second end. The collar body may be securable to the stem portion at a second interface adjacent to the second end in an assembled configuration. The at least one flange may include a ramp adapted to cause the at least one flange deflect outwardly in a first direction in response to engagement with the bone. The at least one flange may be compressible in a second direction against a periphery of the bone subsequent to the deflection.

In any implementations, the second end of the collar body may be adapted to seat the collar portion on bone in the assembled configuration.

In any implementations, the collar body may be securable to a first portion at a first interface adjacent to the first end.

In any implementations, the first portion may be adapted to mount an articulation member. The articulation member may be adapted to mate with an opposed articular surface.

In any implementations, the at least one flange may include a plurality of flanges distributed about a periphery of the collar body.

In any implementations, the at least one flange may extend past the second interface to a free end such that the free end may be axially aligned with the stem body relative to the axis in the assembled configuration.

In any implementations, the ramp may be established adjacent to the free end of the at least one flange.

In any implementations, the at least one flange may include an aperture dimensioned to receive a fastener such that the fastener may apply a compressive force that may cause the at least one flange to move inwardly towards the stem body. The stem body may include a stem passage spaced apart from the collar portion in the assembled configuration. The stem passage may be dimensioned to receive a portion of the fastener.

In any implementations, the collar portion may include a separation gap between an inner surface of the at least one flange and a periphery of the collar body.

In any implementations, the system may include at least one trial that may have a main body and at least one flange extending from the main body. The at least one trial may be dimensioned to represent the collar portion.

A system for an orthopaedic procedure according to an implementation may include a head portion adapted to mount an articulation member. The articulation member may be adapted to mate with an opposed articular surface. A stem portion may be insertable into bone. The stem portion may include a stem body extending along an axis. A collar portion may include a collar body adapted to interconnect the head portion and the stem portion in an assembled configuration. The collar portion may include a plurality of protrusions extending from a distal end of the collar body. The protrusions may be engageable with bone to limit relative rotation between the collar portion and the bone.

In any implementations, the collar body and the stem body may establish a taper connection in the assembled configuration.

A method of installing an orthopaedic implant system according to an implementation may include coupling a stem portion and a collar portion. The collar portion may include at least one flange. The method may include inserting the stem portion into an intramedullary canal of a bone. The method may include trapping a cortical wall of the bone between the at least one flange and the stem portion in response to compressing the at least one flange against the bone. The method may include coupling a head portion and the collar portion such that the collar portion may interconnect the stem portion and the head portion. The method may include mounting an articulation member to the head portion. The articulation member may mate with an opposed articular surface.

In any implementations, the compressing step may include compressing an engagement surface of the at least one flange against the cortical wall. The least one flange may include a ramped engagement surface that may slope outwardly from the engagement surface. The step of coupling the stem portion and the collar portion may include engagement between the ramped engagement surface and the cortical wall such that the at least one flange may deflect outwardly prior to trapping the cortical wall.

In any implementations, the method may include resecting the bone to establish a resection surface along a resection plane that may extend across the cortical wall and intersects the intramedullary canal. the step of coupling the stem portion and the collar portion may occur such that a distal face of the collar portion may be seated on the resection surface.

In any implementations, the method may include selecting a trial that may be representative of the collar portion. The method may include placing the trial relative to the resected bone. The method may include determining a fit of the trial relative to the resected bone. The method may include selecting the collar portion based on the determined fit.

In any implementations, the trial may include a main body and a flange that may extend from the main body. The flange may be offset from an axis of the main body by an offset distance. The offset distance may be associated with an amount that may be sufficient for the least one flange of the collar portion to establish a compressive force against the cortical wall of the bone.

In any implementations, the compressing step may include positioning a fastener through an aperture in the at least one flange, then through the cortical wall, and then into a passage in the stem portion.

In any implementations, the compressing step may include looping a flexible construct about the at least one flange and a periphery of the cortical wall. The compressing step may include tensioning the flexible construct to compress the at least one flange against the cortical wall.

In any implementations, the flexible construct may include a length of suture or a length of wire.

In any implementations, the coupling step may include establishing a taper connection between the collar portion and the stem portion.

In any implementations, the bone may be a humerus. The cortical wall may be associated with a shaft of the humerus.

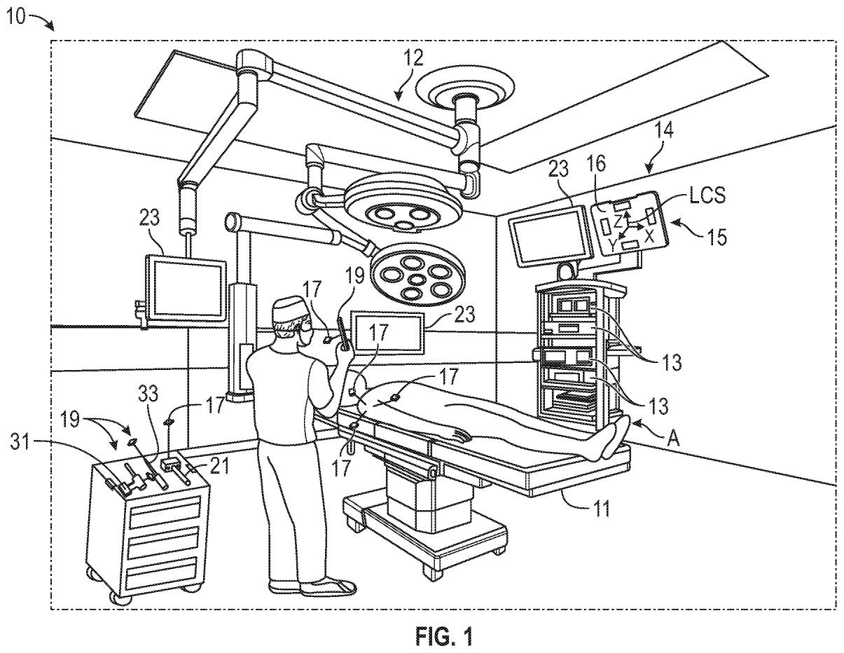

FIG. 1 discloses a surgical suite (e.g., system or assembly) 10 according to an implementation. The surgical suite 10 may be utilized to perform various surgical procedures, including an orthopaedic procedure such as an arthroscopy or an arthroplasty to restore functionality to one or more bones and/or joints. The surgical procedure may include repair of a glenoid during a shoulder reconstruction, including a total shoulder arthroplasty (TSA) or reverse shoulder arthroplasty (RSA). The suite 10 may be utilized in the repair of other locations of the anatomy and other surgical procedures including repair of other bones and joints such as the foot, ankle, wrist, hand, hip, spine and knee.

The suite 10 may include an operating table 11 for supporting an anatomy A of a patient. The suite 10 may include a light assembly 12, which may include one or more light sources for communicating light towards the patient anatomy A.

The suite 10 may include one or more computing devices 13. The computing device(s) 13 may include (e.g., processing) circuitry, including one or more processors coupled to memory, input devices, and/or output devices. The processor(s) may be collectively operable to perform any of the functionality disclosed herein. The computing device(s) 13 may be operable to establish a surgical plan and/or implement the surgical plan for treating the patient.

The suite 10 may include an equipment tower 14. The equipment tower 14 may include one or more modules (e.g., systems), which may incorporate the computing device(s) 13. The module(s) may include a guidance (e.g., navigation or tracking) module 15. The guidance module 15 may include a localizer 16, which may be operatively coupled to the computing device(s) 13. The localizer 16 may include a sensor unit having one or more sensors. One or more trackers 17 may be situated (e.g., fixed or secured) relative to the patient anatomy A. The trackers 17 may comprise an anatomy tracker. The trackers 17 may include one or more objects (e.g., markers). The trackers 17 may include active devices (e.g., sensors or light emitting diodes) and/or passive devices (e.g., reflectors). The tracker(s) 17 may be placed relative to the anatomy A and/or one or more surgical devices (e.g., instruments) 19. The devices 19 may include any of the devices disclosed herein. The devices 19 may include one or more surgical guides 21, cutting instruments (e.g., saw, drill, rasp or reamer) 31 and/or surgical probes 33. The tracker(s) 17 may be placed relative to one or more landmarks of the anatomy A. The surgeon or clinical user may manipulate the surgical devices 19 during a surgical procedure. The localizer 16 may be operable to determine (e.g., track) the position and/or orientation of the trackers 17. The guidance module 15 may be operable to determine the position and/or orientation of each tracker 17 with respect to a (e.g., localizer or global) coordinate system (e.g., framework) LCS of the localizer 16. The suite 10 may be operable to transfer coordinates in the coordinate system LCS to another coordinate system (e.g., framework), such as a local coordinate system of a surgical device 19 or a coordinate system associated with a surgical planning system, and/or vice versa, using various transformation techniques.

The suite 10 may include one or more displays 23. The computing device(s) 13 may be operable to cause the display(s) 23 to display various data and/or information associated with a patient, including a surgical plan and/or guidance information. A surgeon or clinical user may interact with the display(s) 23. The guidance module 15 may be operable to cause the display(s) 23 to display a position and/or orientation of the tracker(s) 17 and/or associated surgical device(s) 19 relative the anatomy A of the patient, including relative one or more anatomical (e.g., bone) models associated with the anatomy A. The computing device(s) 13 may be operable to determine whether the surgical device(s) 19 are positioned and/or oriented relative to the anatomy A according to the surgical plan.

The surgical plan may include various information for treating the anatomy of a patient, including any of the information disclosed herein. The surgical plan may include instrumentation including cutting instruments and/or trials, revision(s) to the anatomy including one or more resection planes and/or reaming depths, and implant(s) including type, size and/or placement relative to the anatomy.

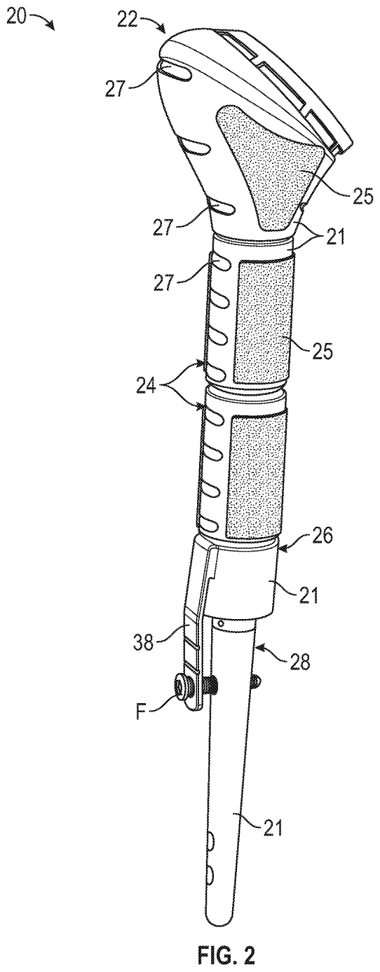

FIGS. 2-8 disclose an orthopaedic implant system (e.g., assembly) 20 according to an implementation. The system 20 may be utilized in various orthopedic procedures to restore functionality to bones and/or joints such as a shoulder joint during an anatomical or reverse shoulder arthroplasty. The system 20 may be utilized in a reconstruction procedure and may be utilized to restore functionality to one or more bones having bone loss and/or which may be in a fractured or shattered condition. The bone loss may be due to loosening of a previously implanted prosthesis. The system 20 may be utilized to in a reconstruction procedure and may be utilized to restore functionality to long bones such as a humerus or femur. The system 20 may be utilized to restore functionality to other bones and joints such as a knee, hip, elbow, wrist or ankle.

Referring to FIG. 2, the system 20 may include one or more components (e.g., portions) 21. The components 21 may be integrally formed or may be separate and distinct components that may be fixedly attached or otherwise secured to each other. The components 21 may be provided as a kit. The components 21 may be provided in various shapes and/or sizes to treat different patient anatomies. The surgeon or assistant may select various components 21 from the kit to establish the system 20.

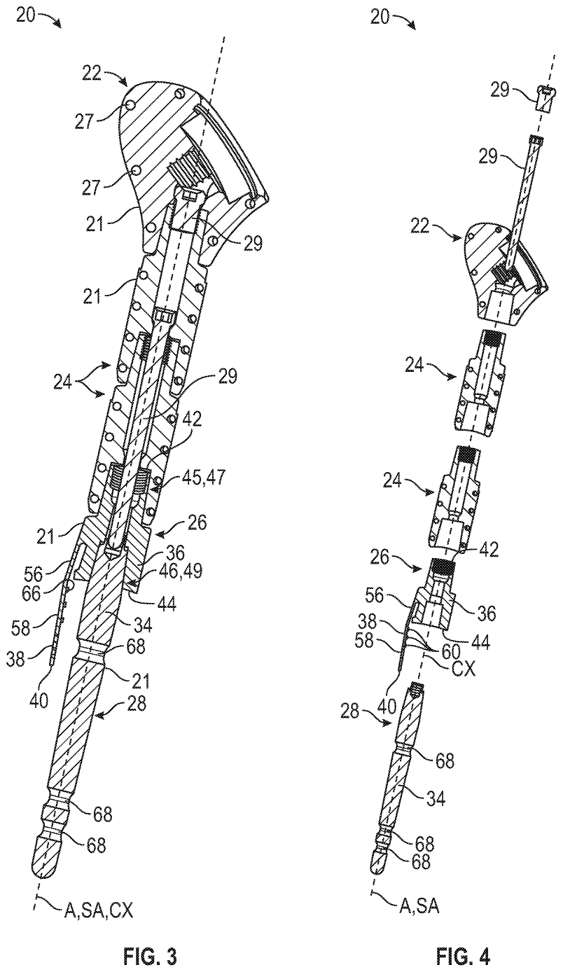

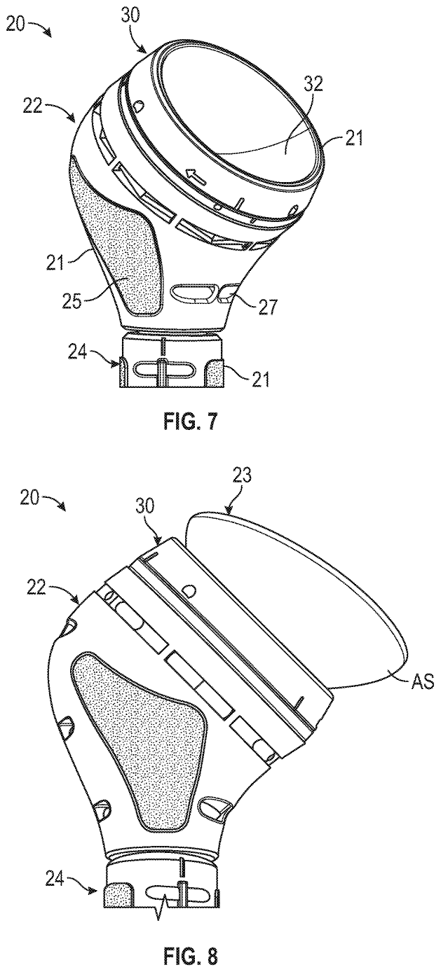

Referring to FIGS. 3-5, with continuing reference to FIG. 2, the components 21 may include a first (e.g., head, cup or proximal) portion 22, one or more second (e.g., spacer, segment or extension) portions 24, a third (e.g., collar) portion 26, and/or a fourth (e.g., stem or distal) portion 28. The head portion 22 may be adapted to mount an articulation member (e.g., liner) 30 (e.g., FIGS. 7-8). The articulation member 30 may include an articulation surface 32 (e.g., FIG. 7). The articulation surface 32 may include various geometries, such a substantially convex geometry or a substantially concave geometry. In implementations, the articulation surface 32 may be adapted to mate with an opposed articular surface AS (e.g., FIG. 8). The articular surface AS may be associated with an adjacent bone or an adjacent implant 23 of a respective joint. The articular surface AS may be associated with a glenoid of the shoulder joint.

The components 21 may be coupled to each other utilizing various techniques. The components 21 may be coupled to each other utilizing a (e.g., Morse) taper connection and/or one or more fasteners 29. The system 20 may be established by one or more taper connections between the adjacent components 21, including portions 22, 24, 26 and/or 28. The surgeon may impact the component 21 with an instrument (e.g., mallet) to establish the taper connection. The taper connection may establish an interference fit that may serve to limit relative axial and/or rotational movement between the components 21. The fastener(s) 29 may serve to limit relative axial and/or rotational movement between the components 21. Various fasteners may be utilized, including threaded bolts, pins, rivets and clips.

The components 21 may include various features to interact with surrounding bone or other tissue, including one or more surface treatment(s) 25 (e.g., FIG. 2). The surface treatments 25 may be established along one or more surfaces of the components 21. Various surface treatments may be utilized, including a porous construct or scaffold established in a thickness of the component 21 or one or more porous layers of material disposed on a surface of the component 21. The surface treatment may promote fixation with adjacent bone or other tissue. The components 21 may include one or more passages (e.g., eyelets) 27. Each passage 27 may be dimensioned to receive a length of suture to secure bone and/or soft tissue to the system 20. In implementations, the surface treatments 25 and/or passages 27 may be omitted.

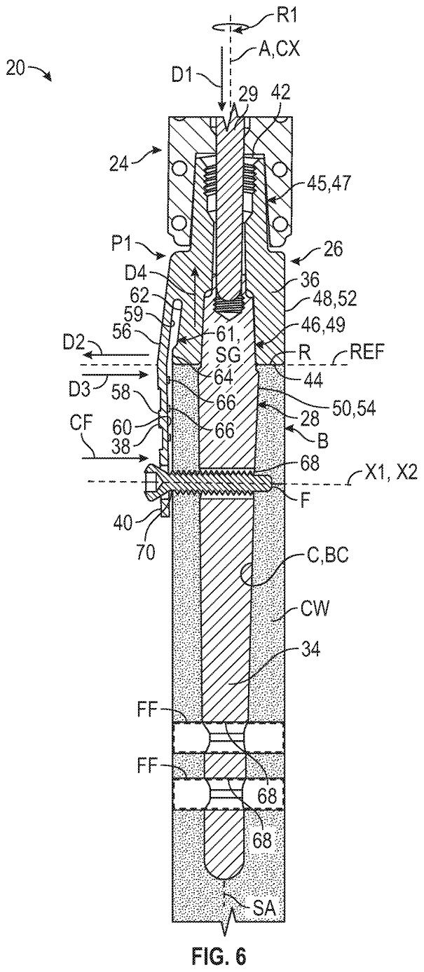

The stem portion 28 may be insertable into bone. In the implementation of FIG. 6, the stem portion 28 may be at least partially insertable in an intramedullary canal C of a (e.g., long) bone B, such as a humerus.

The stem portion 28 may be press fit (e.g., impacted) into the canal C. In implementations, the stem portion 28 may be press fit into a cavity BC formed along the canal C and/or in the bone B. The stem portion 28 may include a stem body 34, which may extend along a stem axis SA and/or an (e.g., longitudinal or main) axis A of the system 20. The stem axis SA may be collinear with, or may be otherwise substantially parallel to, the axis A in an assembled configuration. In implementations, the head portion 22 may be omitted. The system 20 may include a pair of stem portions 28, which may be associated with respective (e.g., adjacent) bone portions and which may be interconnected by the collar portion 26. The implant system 20 may include a set of spacer portions 24 of various geometries (e.g., lengths) to position the head portion 22 and the stem portion 28 relative to each other. In implementations, the spacer portions 24 may be omitted, and the collar portion 26 may be mounted or otherwise secured directly to the head portion 22.

The collar portion 26 may include a collar body 36. The collar body 36 may be adapted to interconnect the stem portion 28 and another component (e.g., portion) 21 of the system 20, such as the head portion 22, in an assembled configuration. The collar body 36 may extend between a first (e.g., proximal) end 42 and a second (e.g., distal) end 44. The collar body 26 may be securable to a first (e.g., adjacent) portion 21 of the system 20 at a first (e.g., proximal) interface 45. The first portion 21 may be a spacer portion 24 or the head portion 22. The collar body 36 may establish the proximal interface 45 with the spacer portion 24 adjacent to the first end 42 in the assembled configuration. In implementations, the collar body 36 and the spacer portion 24 (or head portion 22) may cooperate to establish a (e.g., Morse) taper connection 47 in the assembled configuration. The collar body 26 may be securable to the stem portion 28 at a second (e.g., distal) interface 46, which may be adjacent to the distal end 44 of the collar body 26 in the assembled configuration. The collar body 36 may establish the interface 46 with the stem body 34 adjacent to the distal end 44 in the assembled configuration. The collar body 36 and the stem body 34 may cooperate to establish a (e.g., Morse) taper connection 49 in the assembled configuration.

The collar portion 26 may be dimensioned such that the collar body 36 may be seated on (e.g., against) an associated bone, which may improve a stability of the system 20 when implanted in the patient. The distal end 44 of the collar body 36 may be adapted to seat the collar portion 26 on the bone in the assembled configuration. In the implementation of FIG. 6, the collar body 36 may include a (e.g., collar) periphery 48. The stem body 34 may include a (e.g., stem) periphery 50. The collar periphery 48 and/or stem periphery 50 may have a cylindrical or tapered geometry. The collar periphery 48 may be associated with a (e.g., collar) perimeter 52. The stem periphery 50 may be associated with a (e.g., stem) perimeter 54. The collar perimeter 52 at, or otherwise adjacent to, the distal end 44 may be greater than the stem perimeter 54 at a position aligned with the distal end 44 in the assembled configuration such a portion of the distal end 44 may be adapted to sit on a (e.g., resected) bone surface R of the bone B in the assembled configuration. The resected bone surface R may have a substantially planar geometry and may be established along a resection plane REF. A portion of the bone B may be resected to establish the resected bone surface R, such as a portion of a proximal humerus and/or humeral shaft. The system 20 may be moved in a first (e.g., insertion) direction D1 to position the stem portion 28 at least partially in the intramedullary canal C of the bone B, which may occur subsequent to the resection.

The collar portion 26 may include one or more retention features useful for securing the system 20 to the anatomy. The collar portion 26 may include at least one (e.g., deflectable) flange 38, which may be adapted to engage an adjacent bone. The collar portion 26 may include any number of flanges 38 in accordance with the teachings disclosed herein. The collar portion 26 may include a single flange 38. The flange 38 may be dimensioned to extend from the collar body 36 to a free (e.g., terminal) end 40. The flange 38 may be dimensioned to abut a bone B (e.g., FIG. 6). The free end 40 of the flange 38 may be aligned with the stem body 34 relative to the axis A in the assembled configuration. The flange 38 may be dimensioned such that the free end 40 may be distal to the distal end 44 of the collar body 36 in the assembled configuration. The flange 38 may be dimensioned to extend (e.g., distally) past the second interface 46 to the free end 40 such that the free end 40 may be axially aligned with the stem body 34 relative to the axis A in the assembled configuration. In implementations, the flange 38 may be dimensioned to extend from the distal end 44 of the collar body 36.

The flange 38 may include various geometries. Referring to FIG. 6, with continuing reference to FIGS. 3-5, the flange 38 may include a first (e.g., proximal) flange segment 56 and a second (e.g., intermediate or distal) flange segment 58. The collar portion 26 may include a separation gap SG between an inner surface 59 of the flange 38 and the collar periphery 48. In other implementations, the separation gap SG may be omitted. The first flange segment 56 may be dimensioned to extend radially outward in a second direction D2 from a first position P1 along the collar periphery 48, which may establish a portion of the separation gap SG between the first flange segment 56 and the collar periphery 48. The second flange segment 58 may extend radially inwardly in a third direction D3 from the first flange segment 56 towards the free end 40 to establish an engagement (e.g., inner) surface 60. The second direction D2 and the third direction D3 may be relative to a collar axis CX of the collar body 36 and/or the axis A of the system 20. The collar axis CX may be collinear with, or may be substantially parallel to, the axis A in the assembled configuration. In implementations, the second flange segment 58 may be dimensioned to slope (e.g., flare) inwardly from the first flange segment 56. The engagement surface 60 may be dimensioned to face towards the stem portion 28 in the assembled configuration. The engagement surface 60 may have various geometries. The engagement surface 60 may have a substantially planar or curvilinear contour. The flange 38 may be deformable to follow a contour of an associated bone. In implementations, the engagement surface 60 may have a patient-specific contour that may be dimensioned to follow a contour of an associated bone of a respective patient, which may improve engagement between the flange 38 and the bone.

The flange 38 may be established at various positions along the collar body 36. The first position P1 of the first flange segment 56 may be spaced apart from the proximal end 42 and the distal end 44 of the collar body 36. The separation gap SG may be established by a relief 61 in the collar portion 26. The relief 61 may extend in a fourth (e.g., proximal) direction D4 from the distal end 44 and may be bounded by a portion of the flange 38 joined to the collar body 36. In implementations, the collar portion 26 may include at least one recess 62 in the collar periphery 48. The recess 62 may establish a portion of the relief 61. The recess 62 may extend distally from the first position P1 and/or at least partially along the separation gap SG. The separation gap SG may facilitate flexing of the flange 38 relative to the collar body 36, including deflecting and/or compressing the flange 38. The separation gap SG may be sized to provide a (e.g., preselected) amount of tension on an adjacent bone.

The flange 38 may include one or more engagement features which may improve engagement with an adjacent bone surface. In implementations, the engagement surface 60 may include more engagement features (e.g., protrusions or teeth) 66. The engagement features 66 may be dimensioned to engage (e.g., grip) the bone B to limit relative rotation between the collar portion 26 and the bone B. The engagement features 66 may be distributed about the collar axis CX. The engagement features 66 may include various geometries, such as pyramidal or conical-shaped spikes, spades, and rectangular blades. Other geometries may include granules that establish a texture along the engagement surface 60. Any number of engagement features 66 may be utilized. The engagement features 66 may be uniformly or non-uniformly distributed along the collar portion 26. In other implementations, the protrusions 66 may be omitted.

The flange 38 may be adapted to deflect inwardly toward the collar body 36 and/or collar axis CX in response to a compressive force (e.g., load) CF. The collar body 36 may include at least one abutment 64. The abutment 64 may be dimensioned to bound the recess 62. The abutment 64 may be dimensioned to abut the first flange segment 56 to limit (e.g., radially) inward movement of the flange 38 relative to the collar axis CX and/or axis A, which may limit the amount of compressive force CF applied to a periphery of the bone B. The compressive force CF may have a major component in a direction that may be substantially perpendicular to the axis A.

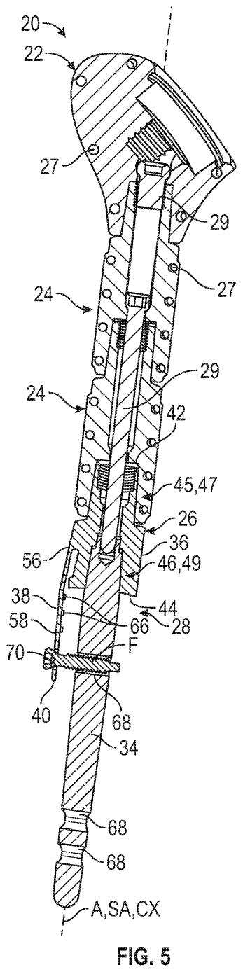

Various techniques may be utilized to establish the compressive force CF. In the implementation of FIG. 6, the flange 38 may include one or more (e.g., flange) apertures 70. The flange aperture 70 may be dimensioned to receive a respective fastener F, which may establish the compressive force CF (see also FIGS. 2 and 5). The fastener F may apply a compressive force CF that may cause the flange 38 to move (e.g., radially) inwardly towards the stem body 34. The stem portion 28 may include one or more (e.g., stem) passages 68. The stem passages 68 may be spaced apart from the collar portion 26 in the assembled configuration. The flange aperture 70 may be at least partially aligned with a respective one of the stem passages 68 in the assembled configuration. A respective one of the stem passages 68 may be dimensioned to receive a portion of the fastener F in the assembled configuration. In implementations, the stem passage 68 may extend along a first axis X1. The flange aperture 70 may be dimensioned to be extend along a second axis X2. The collar portion 26 and stem portion 28 may be positioned relative to each other such that the axes X1, X2 may be co-linear or may otherwise be substantially parallel to each other. The surgeon or assistant may rotate (e.g., swivel) the collar portion 26 in a first rotational direction R1 about the stem portion 28 to position the collar portion 26 and stem portion 28 relative to each other. One or more of the stem passages 68 may be dimensioned to receive a respective fixation fastener FF to secure the stem portion 26 to the bone B. The fasteners F and/or FF may be adapted to extend at least partially or completely through a cortical wall CW of the bone B to secure the system 20 to the bone B.

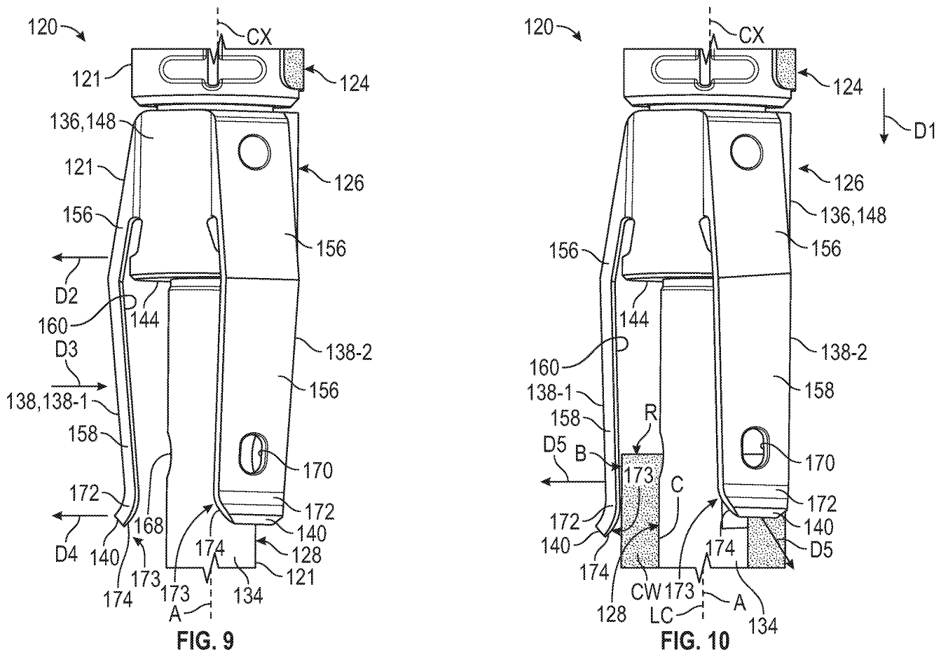

FIGS. 9-11 disclose an orthopaedic system (e.g., assembly) 120 according to another implementation. In this disclosure, like reference numerals designate like elements where appropriate and reference numerals with the addition of one-hundred or multiples thereof designate modified elements that are understood to incorporate the same features and benefits of the corresponding original elements. The system 120 may include a collar portion 126 and/or a stem portion 128.

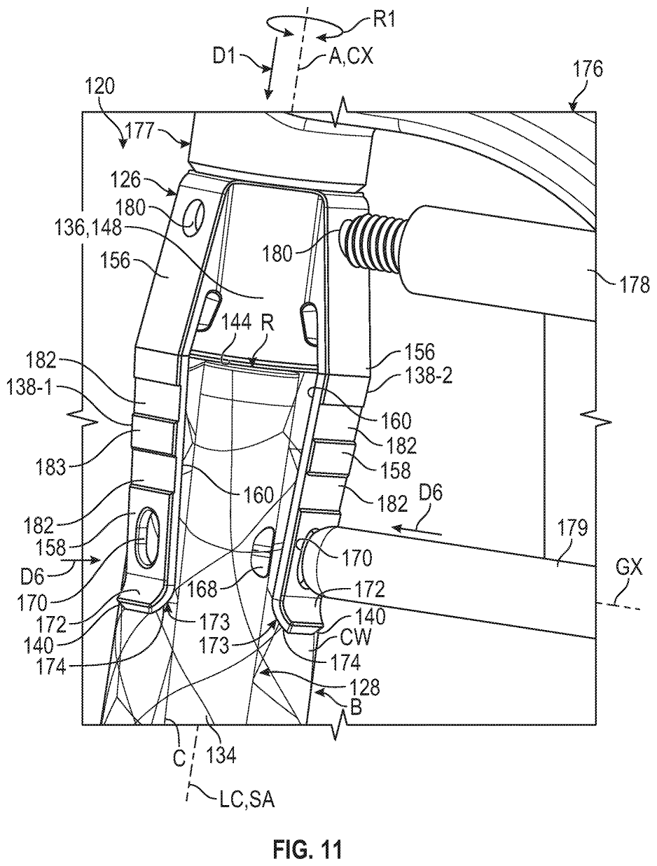

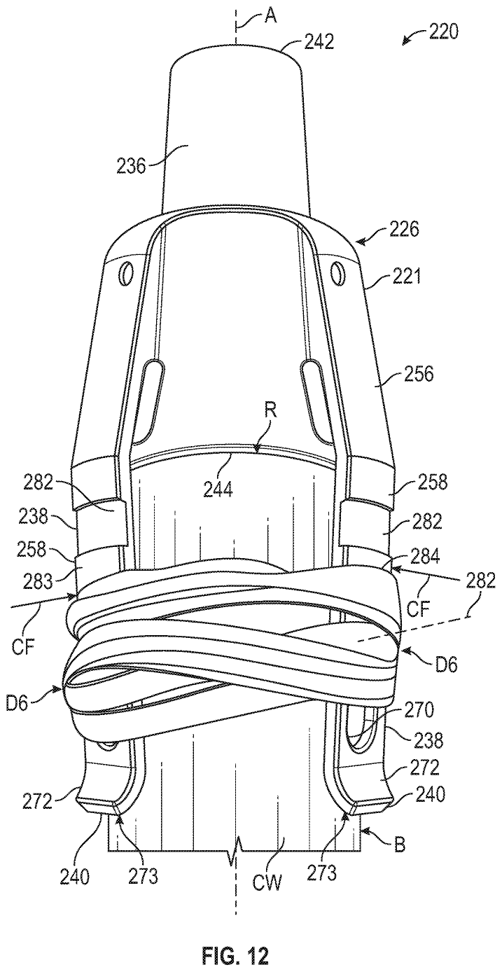

The collar portion 126 may include a collar body 136 and a plurality of flanges 138. The flanges 138 may be distributed about a periphery 148 of the collar body 136. The flanges 138 may include first and second flanges 138-1, 138-2. The flanges 138-1, 138-2 may extend from different positions along the collar body 136. The flanges 138-1, 138-2 may be circumferentially distributed about a collar axis CX of the collar body 136, which may establish a dual flange arrangement. The flanges 138-1, 138-2 may be circumferentially offset by an offset angle relative to the collar axis CX. The offset angle may be less than or equal to approximately 180 degrees, such as between approximately 45 degrees and approximately 120 degrees. In the implementation of FIGS. 9-11, the offset angle may be approximately 90 degrees. In another implementation, the offset angle may be approximately 120 degrees (e.g., FIG. 12). For the purposes of this disclosure, the terms “about,” “approximately” and “substantially” mean±10 percent of the stated value or relationship unless otherwise indicated. The surgeon may position the flanges 138-1, 138-2 circumferentially relative to the stem portion 128 and/or axis A to establish a circumferential direction of a compressive force applied to an associated bone B (e.g., FIGS. 10-11). In the implementation of FIG. 11, the surgeon or assistant may rotate the collar portion 126 in a first rotational direction R1 relative to the axis A to position the collar portion 126 relative to the stem portion 128.

Each flange 138 may include a first (e.g., proximal) flange segment 156, a second (e.g., intermediate) flange segment 158, and/or a third (e.g., distal) flange segment 172. The second flange segment 158 may interconnect the first and third flange segments 156, 172. The second flange segment 158 may include an engagement surface 160, which may be dimensioned to abut a periphery of an adjacent bone B (e.g., FIG. 11). The third flange segment 172 may establish a free end 140 of the flange 138.

The flange 138 may include one or more inflections (e.g., undulations). In other implementations, the flange 138 may be substantially linear. In the implementation of FIG. 9, the first flange segment 156 may be dimensioned to extend (e.g., radially) outwardly in a second direction D2. The second flange segment 158 may be dimensioned to extend (e.g., radially) inwardly in a third direction D3. The third flange segment 172 may be dimensioned to extend (e.g., radially) outwardly in a fourth direction D4. The directions D2, D3, D4 may be relative to the collar axis CX and/or axis A. The directions D2, D4 may differ from each other. In implementations, the third flange segment 172 may be dimensioned to slope (e.g., flare) outwardly from the second flange segment 158.

The flange 138 may include a geometry that may assist in positioning the system 120 relative to a bone, such as during insertion of the stem portion 128 in an intramedullary canal C of the bone B (e.g., FIGS. 10-11). The third flange segment 172 may include a ramp 173, which may be adapted to cause the flange 138 to deflect outwardly in response to engagement with the bone B. The outward deflection may be utilized to accommodate various bone geometries engageable by the flange 138. The ramp 173 may be established adjacent to the free end 140 of the flange 138. The ramp 173 may include a ramped engagement surface 174 that may slope (e.g., flair) outwardly from the engagement surface 160 of the second flange segment 158. The ramped engagement surface 174 may be dimensioned to cause the flange 138 to deflect outwardly in response to engagement with the bone B. The deflection may be in a fifth direction D5, which may occur in response to moving the system 120 in a first (e.g., insertion) direction D1 (e.g., FIG. 10). The ramp 173 may be adapted to cause the flange 138 to deflect outwardly in the fifth direction D5 in response to engagement with the bone B. The insertion direction D1 may be collinear with, or may be substantially parallel to, a longitudinal axis LC of the intramedullary canal C of the bone B. In the implementation of FIG. 11, the flange 38 may be dimensioned such that the ramped engagement surface 174 may be spaced apart from the bone B when the engagement surface 160 may be in abutment with the periphery of the bone B. The flange 138 may be compressible in a sixth direction D6 against a periphery of the bone B subsequent to the deflection, which may occur in response to securing the flange 138 with one or more fasteners to the bone B (e.g., FIG. 6).

Various techniques may be utilized to position fastener(s) relative to the system 120 (e.g., fastener F of FIG. 6). In the implementation of FIG. 11, the collar portion 126 may be adapted to engage a surgical instrument (e.g., targeting guide) 176. The surgical instrument 176 may include a mounting portion 177, an indexing portion 178 and/or a guide portion 179. The mounting portion 177 may be adapted to mount the instrument 176 to the collar body 136. The surgeon or assistant may rotate (e.g., swivel) the mounting portion 177 in the rotational direction R1 about the collar axis CX to set a position of the guide portion 178 relative to the collar portion 126 and/or stem portion 128. The indexing portion 178 may cooperate with a (e.g., selectable) index feature (e.g., depression) 180 along the collar portion 126, which may set a circumferential position of the instrument 176 relative to the collar portion 126 and/or axis A. The guide portion 179 may be cannulated and may be adapted to guide one or more instruments such as a drill bit for forming (e.g., pilot) hole(s) in the bone B. The guide portion 179 may extend along a guide axis GX. A projection of the axis GX may intersect an aperture 170 associated with the respective index feature 180 and/or stem passage 168 when the instrument 176 may be coupled to the collar portion 126. A fastener may be positioned through the aperture 170, the hole formed in a cortical wall CW and/or another portion of the bone B, and/or at least partially through the passage 168 to secure the flange 138 to the bone B (see also FIG. 6).

FIG. 12 discloses an orthopaedic implant system (e.g., assembly) 220 according to another implementation. A collar portion 226 may include one or more flanges 238. The flange 238 may include one or more recesses 282, which may be established along an outer surface 283 of the flange 238 (see also FIG. 11). The recesses 282 may have various geometries, such as a groove, channel or notch. The recesses 282 may be dimensioned to receive portion(s) of a flexible construct 284.

Various arrangements and materials may be utilized to establish the flexible construct 284. The flexible construct 284 may include metallic and/or non-metallic materials, including inorganic and/or organic materials, and combinations thereof. The flexible construct 284 may be established by a wire, chain, band, cable, rope, cord, suture, tape, filaments, yarns and/or adjustable knotless suture/button constructs. The material may be re-shapable and may rebound or may be permanently deformable. In implementations, the flexible construct 284 may be a length of wire or a length of suture, which may have one or more filaments. The filaments may be continuous or discontinuous. The filaments may be braided or twisted together to establish a length of the flexible construct 284. The flexible construct 284 may be continuous or may have a discreet pair of ends securable to each other utilizing a knot or other fixation technique. The flexible construct 284 may be a tensionable loop dimensioned to establish a loop about a collar axis CX of the collar portion 226 in the assembled configuration. The flexible construct 284 may be tied in a knot to form a loop.

The flexible construct 284 may be dimensioned to loop about the axis A of the system 220 and/or collar axis CX to establish compressive force(s) CF. The compressive force(s) CF may be applied to the flange(s) 238 and the associated bone B, which may cause the flanges 238 to move in a sixth direction D6 towards the stem portion 228. In implementations, the flexible construct 284 may be utilized in combination with fastener(s) through aperture(s) 270 in the respective flange(s) 238 (see also FIG. 6) to compress the flange 238 and/or secure the flange 238 to the bone B.

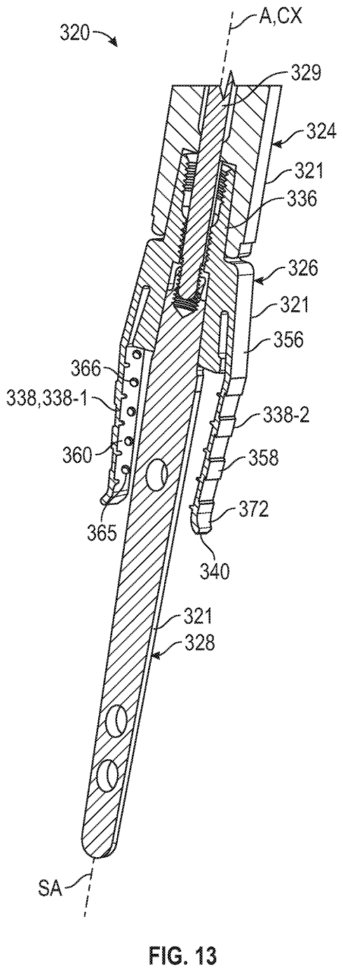

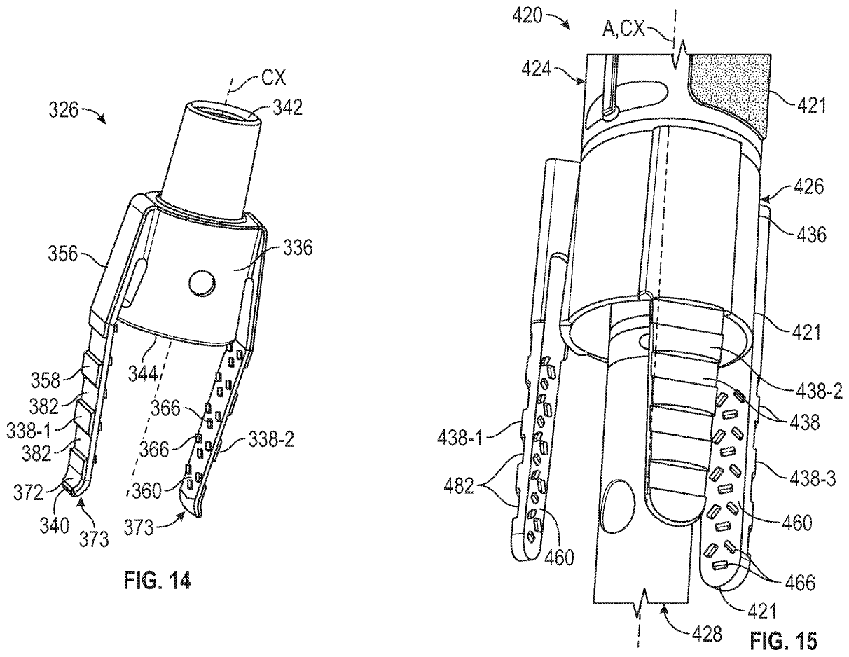

FIGS. 13-14 disclose an orthopaedic implant system (e.g., assembly) 320 according to an implementation. A collar portion 326 may include a set of flanges 338, which may extend from a collar body 336. The flanges 338 may include first and second flanges 338-1, 338-2 on opposite sides of the collar body 336. The flanges 338-1, 338-2 may be circumferentially offset by an offset angle relative to a collar axis CX of the collar body 336 and/or axis A. The offset angle may be approximately 180 degrees. The opposed flanges 338-1, 338-2 may be arranged to apply distributed (e.g., opposing) compressive forces (e.g., loads) on an associated bone.

In the implementation of FIG. 15, a system 420 may include a collar portion 426. The collar portion 426 may include a set of flanges 438-1, 438-2, 438-3 distributed about a collar body 436, which may establish a triple flange arrangement. Offset angles between the adjacent pairs of the flanges 438-1, 438-2, 438-3 may be the same or may differ from each other. The offset angle(s) may be approximately 120 degrees between the adjacent pairs of flanges 438-1, 438-2, 438-3. The flanges 438 may be arranged to distribute compressive forces about a periphery of an associated bone from three associated directions, which may reduce rocking of the system 420 that may otherwise be caused by kinematic and/or static loads associated with the joint.

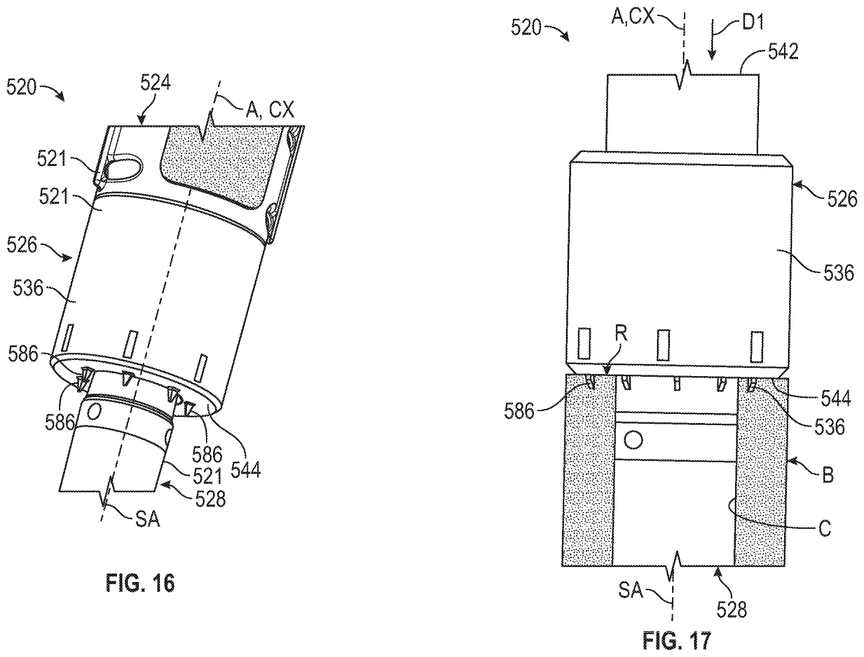

FIGS. 16-17 disclose an orthopaedic implant system (e.g., assembly) 520 according to another implementation. Flanges may be omitted from a collar portion 526. In other implementations, the collar portion 526 may include one or more flanges according to any of the implementations disclosed herein. A collar body 536 of the collar portion 526 may include one or more engagement features (e.g., protrusions) 586. The engagement features 586 may be distributed along a second (e.g., distal) end 544 of the collar body 536. The engagement features 586 may be dimensioned to extend (e.g., distally) from the distal end 544 of the collar body 536. The engagement features 586 may be engageable with bone B to limit relative rotation between the collar portion 526 and the bone B. The engagement features 586 may have various geometries, such as pyramidal or conical-shaped spikes. Other geometries may include planar-shaped blades.

The surgeon may move the collar portion 526 in a first (e.g., insertion) direction D1 relative the bone B, which may cause the collar portion 526 to engage a (e.g., resected) bone surface R of the bone B. The surgeon may move the collar portion 526 in the insertion direction D1 with the stem portion 528 as a unit, or the collar portion 526 may be coupled to the stem portion 528 subsequent to positioning the stem portion 528 in an intramedullary canal C of the bone B. The surgeon may impact the collar portion 526 with an instrument (e.g., mallet) to drive the engagement features 586 into the resected bone surface R. The engagement features 586 may be driven into the bone B until the distal end 544 of the collar body 536 may be seated against the bone surface R.

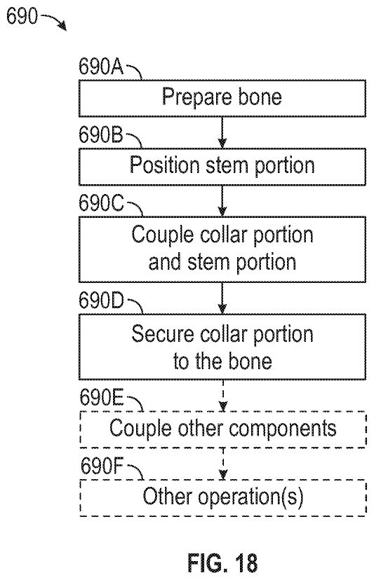

FIG. 18 discloses a method of installing an orthopedic implant system (e.g., assembly) in a flow chart 690 according to an implementation. The method 690 may be utilized in an orthopedic procedure to restore functionality to a bone and/or joint, including any of the bones and joints disclosed herein. The method 690 may be utilized in an anatomical shoulder or reverse shoulder arthroplasty. The method 690 may be utilized to treat bones having bone loss, which may occur due to loosening of a previously implanted prosthesis. In implementations, the method 690 may be utilized in repair of a fractured or shattered proximal humerus or other long bone. The method 690 may be utilized with any of the implant systems disclosed herein, including implant systems 20, 120, 220, 320, 420 and/or 520. Fewer or additional steps than are recited below could be performed within the scope of this disclosure, and the recited order of steps is not intended to limit this disclosure. Reference is made to the implant system 20.

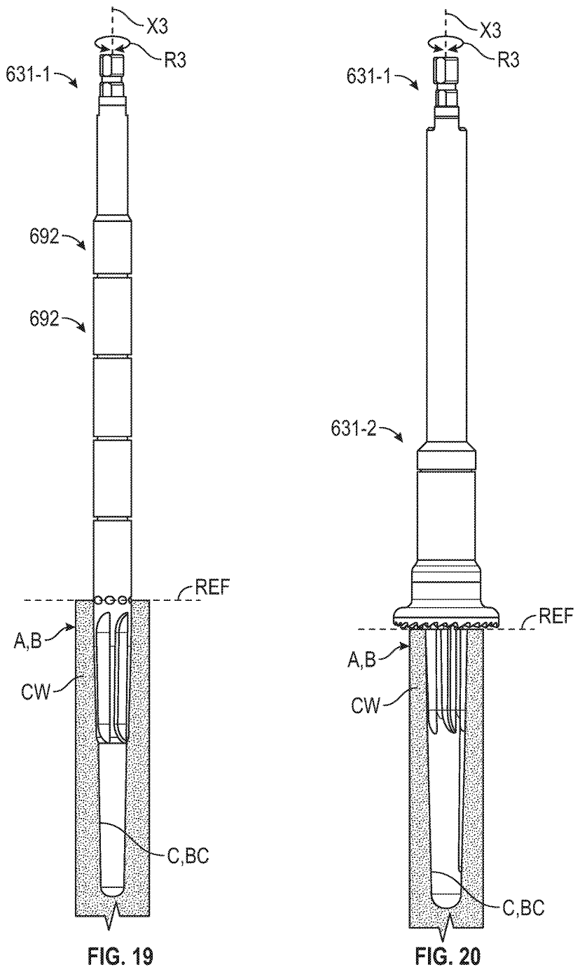

Referring to FIG. 6, with continuing reference to FIG. 18, a bone B may be prepared at block 690A. The bone B may be a long bone, such as a humerus or femur. Block 690A may include removing (e.g., resecting) a portion of the bone B, such as a portion of the proximal humerus or femur. Block 690A may include cutting a portion of the bone B with a cutting tool (e.g., saw blade). The bone B may be resected to establish a resection surface R along a resection plane REF. The resection surface R may be substantially planar. The resection plane REF may extend across a cortical wall CW of the bone B and may intersect an intramedullary canal C of the bone B. The cortical wall CW may be associated with a shaft of a long bone, such as the humerus or femur. Block 690A may include establishing a cavity (e.g., opening) BC along the intramedullary canal C, which may occur subsequent to removing (e.g., resecting) a portion of the bone (e.g., FIG. 19). The cavity BC may be established utilizing various techniques, such as a rasp, drill or milling tool. The cavity BC may be sized to accommodate the stem portion 28.

In the implementation of FIG. 19, the cavity BC may be established by a first cutting instrument 631-1. The first cutting instrument 631-1 may include an (e.g., intramedullary) reamer (e.g., drill). The cutting instrument 631-1 may be rotatable in a third rotational direction R3 about a third axis X3 to remove a portion of the bone B. The cutting instrument 631-1 may include indicia 692, which may be associated with respective positions along the axis X3. The surgeon may use the indicia 692 to establish a distance (e.g., depth) of the cavity BC.

In the implementation of FIG. 20, block 690A may include using a second cutting instrument 631-2 to remove a portion of the bone B. The second cutting instrument 631-2 may include a reamer (e.g., planer). The second cutting instrument 631-2 may be coupled (e.g., fixedly attached) to the first cutting instrument 631-1. The second cutting instrument 631-2 may be releasably securable to the first cutting instrument 631-1. In other implementations, the cutting instruments 631-1, 631-2 may be integrally formed. The surgeon may be provided with cutting instruments 631-1 and/or 631-2 of different shapes and/or sizes. The surgeon may use the second cutting instrument 631-2 to establish (e.g., refine) the resection plane REF. The cutting instrument 631-2 may be rotatable in the third rotational direction R3 about the third axis X3 to remove a portion of the bone B. The second cutting instrument 631-2 may be used to establish the resection plane REF at an orientation that may be substantially perpendicular to a (e.g., longitudinal) axis of the bone B and/or the axis X3 of the instrument 631-1.

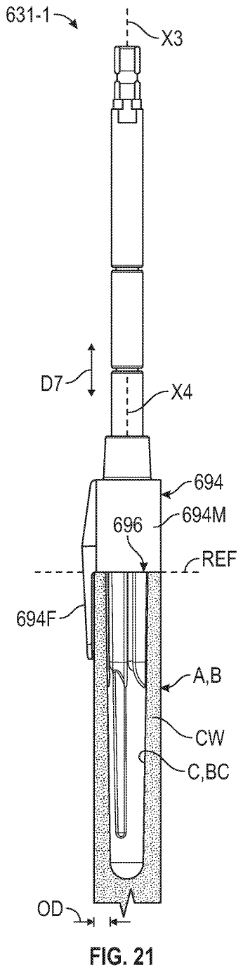

Block 690A may include trialing the anatomy A, which may occur during and/or subsequent to preparing the bone B. In the implementation of FIG. 21, block 690A may include determining a size of the collar portion 26, which may be suitable for treating the anatomy A. The surgeon may be provided with at least one or more trials 694, which may be provided in a surgical kit. The trial 694 may be dimensioned to represent an associated collar portion 26. The kit may include a set of trials 694 that may be associated with a set of collar portions 26, which may have different sizes. The surgeon may select one or more trials 694 from the kit. The system 20 may include the instrument(s) 631 and/or trial(s) 694 (e.g., FIG. 1).

The trial 694 may be movable in a seventh direction D7 along the cutting instrument 631-1, which may be substantially parallel to the third axis X3. The trial 694 may abut the bone B at an interface 696, which may be associated with the resection plane REF.

The trial 694 may include a main body 694M and at least one, or more than one, flange 694F. The main body 694M may include an opening, which may be dimensioned to at least partially receive the instrument 631-1. The trial 694 may be movable (e.g., slidable or translatable) along the instrument 631-1. The flange 694F may extend from the main body 694M. The flange 694F may be substantially rigid. The flange 694F may be offset by an offset distance OD from a fourth axis X4 of the main body 694M. The offset distance OD may be associated with a distance between the associated flange 38 and the collar axis CX of the associated collar 26 (e.g., FIGS. 2-3). The offset distance OD may be an amount sufficient such that the flange 38 may move along and may (e.g., maintain) contact with the cortical wall CW. The offset distance OD may be associated with an amount sufficient to bias the flange 38 in the second direction D2 such that the flange 38 may establish a (e.g., predetermined) compressive force CF against the cortical wall CW of the bone B (e.g., FIG. 6). The offset distance OD may be associated with an amount that may be sufficient for the flange(s) 38 of the collar 26 to establish the (e.g., predetermined) compressive force CF against the cortical wall CW of the bone B.

Block 690A may include selecting a trial 694, which may be representative of an associated collar (e.g., collar portion) 26. Block 690A may include placing the trial 694 relative to the (e.g., resected) bone B. Block 690A may include determining a fit of the trial 694 relative to the resected bone B and may include selecting the collar 26 based on the determined fit.

The surgeon may select a trial 694 having a size that may be less than a diameter of the bone B. The flange 694F may contact the bone B along the resection plane REF, which may preclude the main body 694M from being seated on the bone B to establish the interface 696. The surgeon may select a trial 694 having a size that may be greater than the diameter of the bone B. The main body 694M may abut the bone B at the interface 696, but the flange 694F may be spaced apart from the cortical wall CW of the bone B. The surgeon may select another trial 694 in response to determining an (e.g., inadequate) fit between the trial 694 and the bone B. The surgeon may select a trial 694 having a size that may be substantially equal to a diameter of the bone B. The main body 694M may abut the bone B at the interface 696, and the flange 694F may abut the cortical wall CW of the bone B when the main body 694M may be seated on the bone B at the interface 696. The surgeon may remove the surgical instrument 631-1 and the trial 694. The surgeon may select a collar 26 based on the size of the (e.g., selected) trial 694.

One or more components 21 may be coupled together to establish a system (e.g., assembly) 20. The components 21 may include any of the components disclosed herein and may be provided as a kit. The kit 21 may include components 21 in different shapes and/or sizes. The surgeon or assistant may select components 21 from the kit based on an anatomy of the patient and a pathology of the bone.

At block 690B, the stem portion 28 may be positioned relative to the bone B. Block 690B may include inserting the stem portion 28 into the intramedullary canal C of the bone B. The stem portion 28 may be press fit (e.g., impacted) into the cavity BC of the bone B. The stem portion 28 may be secured to the bone B with one or more fasteners FF, which may be positioned through respective passages 68 in the stem portion 28.

At block 690C, the stem portion 28 and collar portion 26 may be coupled to each other. The collar portion 26 and stem portion 28 may be coupled to each other utilizing any of the techniques disclosed herein, such as a taper connection. A taper connection 49 may be established between the collar portion 26 and the stem portion 28.

The collar portion 26 may include one or more flanges 38. In implementations, the flanges may be omitted (e.g., FIGS. 16-17). Coupling the collar portion 26 and stem portion 28 may occur prior, during and/or subsequent to positioning the stem portion 28 in the bone B at block 690B.

In the implementation of FIG. 11, positioning the collar portion 126 may occur such that the flange(s) 138 may deflect outwardly in response to engagement between a ramp 173 of the respective flange 138 and the bone B. The flange 138 may include a ramped engagement surface 174 that may slope outwardly from an engagement surface 160. The ramped engagement surface 174 may be established along the ramp 173. Positioning the collar portion 126 may cause the ramped engagement surface 174 of the ramp 173 to engage the resection surface R and/or another portion of the bone B. Positioning the collar portion 126, including coupling the collar portion 126 and the stem portion 128, may include engagement between the ramped engagement surface 174 and the cortical wall CW such that the flange 138 may deflect (e.g., radially) outwardly prior to trapping the cortical wall CW between the flange 138 and the stem portion 128.

In the implementation of FIG. 17, the collar portion 526 may be seated against the bone B. The surgeon may impact the collar portion 526 to drive one or more engagement features 586 into the resection surface R and/or another portion of the bone B.

At block 690D, the collar portion 26 may be secured to the bone B. The collar portion 26 may be secured utilizing any of the techniques disclosed herein. Positioning the collar portion 26 may include compressing the flange(s) 38 against the bone B. Compressing the flange 38 may include compressing an engagement surface 60 of the flange 38 against the cortical wall CW of the bone B. The collar portion 26 may be positioned such that a cortical wall CW of the bone B may trapped (e.g., captured) between the flange 38 and the stem portion 28, which may occur in response to compressing the flange 38 against the bone B. Coupling the stem portion 28 and the collar portion 26 may occur such that the distal face 44 of the collar portion 26 may be seated on the resection surface R.

The flanges 38 may be compressed utilizing any of the techniques disclosed herein. A fastener F may be positioned through an aperture 70 in the flange 38 and at least partially into the bone B. A surgical instrument may be utilized to establish a trajectory of the fastener F (e.g., instrument 176 of FIG. 11). In implementations, the fastener F may be positioned through the aperture 70, then through the cortical wall CW of the bone B, and then into a stem passage 68 in the stem portion 28. A (e.g., preselected) amount of torque may be applied to the fastener F to establish a (e.g., preselected) amount of compression on the bone B.

In the implementation of FIG. 12, a flexible construct 284 may be looped about the flange(s) 238 and a periphery of the cortical wall CW of the bone B. The flexible construct 284 may be tensioned to compress the flange(s) 238 against the cortical wall CW of the bone B. The flexible construct may include any of the constructs disclosed herein, such as a length of suture or a length of wire.

At block 690E, one or more components 21 may be coupled to the stem portion 28 and/or collar portion 26 to establish the system 20, including any of the components disclosed herein. A head portion 22 and the collar portion 26 may be coupled such that the collar portion 26 may interconnect the stem portion 28 and the head portion 22. One or more spacer portions 24 may be coupled between the head portion 22 and the collar portion 26, which may establish a (e.g., preselected) height of the head portion 22 relative to the stem portion 28. An articulation member 30 may be coupled (e.g., mounted) to the head portion 22. The articulation member 30 may be arranged to mate with an opposed articular surface of an adjacent bone or implant to restore functionality to a joint.

At block 690F, one or more other operations may be performed, which may occur prior to, during, or subsequent to any of blocks 690A to 690E. The operations may include positioning one or more bone fragments about the system 20 and/or attaching soft tissue to the system 20. One or more incisions in the patient may be closed.

The novel implant systems and methods of this disclosure may provide versatility in repairing bone defects restoring functionality to bones and/or joints. The implant system may include a collar portion including one or more flanges and/or engagement features. The flanges may be adapted to deflect in response to engagement with bone, which may be suitable for accommodating different patient anatomies. The flanges may be adapted to abut an external surface of a bone and may be compressed against the bone to promote torsional stability when the implant system may be placed under torsional loads. Torsional stability may promote alignment and functionality of the restored joint and healing of the patient.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure.

1. A system for an orthopaedic procedure comprising:
a head portion adapted to mount an articulation member, wherein the articulation member is adapted to mate with an opposed articular surface;
a stem portion insertable into bone, wherein the stem portion includes a stem body extending along an axis; and
a collar portion including a collar body and at least one deflectable flange that extends from the collar body to a free end, the collar body is adapted to interconnect the head portion and the stem portion in an assembled configuration, and the at least one flange is dimensioned to abut bone.

2. The system as recited in claim 1, wherein:
the free end is aligned with the stem body relative to the axis in the assembled configuration.

3. The system as recited in claim 1, wherein the at least one flange includes a plurality of flanges distributed about a periphery of the collar body.

4. (canceled)

5. The system as recited in claim 1, wherein:
the collar body extends between a proximal end and a distal end, and the collar body establishes an interface with the stem portion adjacent to the distal end in the assembled configuration.

6. The system as recited in claim 5, wherein:
the free end of the at least one flange is distal to the distal end of the collar body in the assembled configuration.

7. The system as recited in claim 5, wherein:
a perimeter of the collar body at the distal end is greater than a perimeter of the stem body aligned with the distal end such that a portion of the distal end is adapted to sit on a resected bone surface in the assembled configuration.

8. (canceled)

9. The system as recited in claim 1, wherein:
the at least one flange includes a first flange segment and a second flange segment;
the first flange segment extends radially outwardly in a first direction from a first position along a periphery of the collar body to establish a separation gap between the first flange segment and the periphery of the collar body; and
the second flange segment extends radially inwardly in a second direction from the first flange segment towards the free end to establish an engagement surface dimensioned to face towards the stem portion in the assembled configuration, and the first and second directions are relative to the axis.

10. The system as recited in claim 9, wherein:
the at least one flange includes a third flange segment that establishes the free end;
the second flange segment interconnects the first and third flange segments; and
the third flange segment extends radially outwardly in a third direction relative to the axis, the third flange segment includes a ramped engagement surface that slopes outwardly from the engagement surface of the second flange segment, and the ramped engagement surface is dimensioned to cause the at least one flange to deflect outwardly in response to engagement with bone.

11. (canceled)

12. The system as recited in claim 9, wherein:
the collar portion includes a recess in the periphery of the collar body that extends distally from the first position along the separation gap.

13. (canceled)

14. The system as recited in claim 1, wherein the at least one flange is adapted to deflect inwardly toward the collar body in response to a compressive force.

15. The system as recited in claim 14, wherein the at least one flange includes an aperture dimensioned to receive a fastener that establishes the compressive force.

16. The system as recited in claim 15, wherein:
the stem portion includes a stem passage spaced apart from the collar portion in the assembled configuration; and
the stem passage is dimensioned to receive a portion of the fastener in the assembled configuration.

17. The system as recited in claim 14, wherein:
the at least one flange includes one or more recesses dimensioned to receive a flexible construct; and
the flexible construct is dimensioned to loop about the axis to establish the compressive force.

18-19. (canceled)

20. A system for an orthopaedic procedure comprising:
a stem portion insertable into bone, wherein the stem portion includes a stem body extending along an axis; and
a collar portion including a collar body and at least one flange dimensioned to abut bone, wherein the collar body extends between a first end and a second end, the collar body is securable to the stem portion at a second interface adjacent to the second end in an assembled configuration, the at least one flange includes a ramp adapted to cause the at least one flange deflect outwardly in a first direction in response to engagement with the bone, and the at least one flange is compressible in a second direction against a periphery of the bone subsequent to the deflection.

21. The system as recited in claim 20, wherein the second end of the collar body is adapted to seat the collar portion on bone in the assembled configuration.

22. The system as recited in claim 20, wherein the collar body is securable to a first portion at a first interface adjacent to the first end.

23.(canceled)

24. The system as recited in claim 20, wherein the at least one flange includes a plurality of flanges distributed about a periphery of the collar body.

25. The system as recited in claim 20, wherein:
the at least one flange extends past the second interface to a free end such that the free end is axially aligned with the stem body relative to the axis in the assembled configuration; and
the ramp is established adjacent to the free end of the at least one flange.

26. (canceled)

27. The system as recited in claim 20, wherein:
the at least one flange includes an aperture dimensioned to receive a fastener such that the fastener applies a compressive force that causes the at least one flange to move inwardly towards the stem body; and
the stem body includes a stem passage spaced apart from the collar portion in the assembled configuration, and the stem passage is dimensioned to receive a portion of the fastener.

28. (canceled)

29. The system as recited in claim 20, further comprising:
at least one trial including a main body and at least one flange extending from the main body;
wherein the at least one trial is dimensioned to represent the collar portion.

30-42. (canceled)
